(19) United States
(12) Patent Application Publication
(10) Pub. No.: US 2010/0228141 A1
(43) Pub. Date: Sep. 9, 2010

(54) Tamper resistant receptacle where access is actuated by breath samples and method of manufacturing the same

(76) Inventor: Theodosios Kountotsis, East Elmhurst, NY (US)

Correspondence Address: Theodosios Kountotsis, 32-31 72 Street, East Elmhurst, NY, 11370, US

(21) Appl. No.: 12/380,951

(22) Filed: Mar. 5, 2009

(51) Int. Cl. A61B 5/08; B65D 50/00; B65B 7/28; B65D 55/02

(52) U.S. Cl. 600/532; 220/260; 53/485; 206/1.5; 600/543

(57) ABSTRACT

A sealing apparatus including an inlet for receiving a first breath sample from a user for fastening the sealing apparatus to an external device, the first breath sample including a plurality of organic compounds or molecules, a select combination of the plurality of organic compounds or molecules being used to create a unique breath profile; and a module for analyzing and storing the first breath sample as the unique breath profile; wherein the inlet further receives a second breath sample and compares the second breath sample with the first breath sample to determine whether access to the external device is permitted.

### BACKGROUND

1. Field of the Related Art

The present disclosure relates to medical containers/vials/receptacles, and more particularly, but not exclusively, to a tamper-resistant medical container/vial/receptacle whose access is actuated by breath samples for preventing unauthorized uses.

2. Description of the Related Art

Drug abuse is a very serious problem in the United States, as well as several other countries around the world. Some drugs are illegal and some drugs are legal. For example, marijuana, cocaine, heroin, crack, meth, ecstasy, steroids, etc. are illegal drugs that cannot be obtained with a prescription from a doctor. However, legal drugs can be obtained with a prescription from a doctor. There are a multitude of drugs that can be obtained legally from a doctor. These are referred to as prescription drugs. Prescription drugs can include opioids, anti-depressants, stimulants, painkillers, sedatives, and any other types of over-the-counter prescription drugs available at any pharmacy with a doctor's approval.

Recently, especially teenagers, but other adults too, that have not been prescribed a certain medication have been abusing prescription drugs. In fact, many of today's teens are more likely to abuse prescription (Rx) and over-the-counter (OTC) medications than many illegal drugs and such teens believe that abusing medicines to get high is ‘safer’ than using illegal drugs. The intentional abuse of prescription (Rx) and over-the-counter (OTC) medications to get high is now an entrenched behavior among today's teen population, according to a national study released by the Partnership for a Drug-Free America®. In a 2006 2006 study conducted by the Partnership for a Drug-Free America®, nearly one in five teens (19% or 4.5 million) report abusing prescription medications to get high and 1 in 10 (10% or 2.4 million) report abusing cough medicine to get high. According to a 2007 survey conducted for the National Institute on Drug Abuse, prescription drug abuse occurs in more than 15% of U.S. high school seniors. The types of drugs most popular for prescription drug abuse are codeine-based painkillers such as oxycodone (OxyContin) and those containing hydrocodone (Vicodin). In 2007, a total of 5.2 million people aged 12 and older (including 1.5 million young adults) said they had misused prescription pain relievers in the past month, according to the report, which is based on a series of nationwide surveys. Needless to say, these statistics of teenage drug abuse, and prescription drug abuse in general, are disturbing and horrific.

Several other studies also found that teens believe a key driver for abusing prescription drugs is their widespread availability and easy access. According to the data, more than three in five teens say Rx pain relievers are easy to get from parents' medicine cabinets. Half of teens say they're easy to get through other people's prescriptions and more than half of teens say pain relievers are “available everywhere.” Additionally, 43% of teens believe pain relievers are cheap and 35% believe they are safer to use than illegal drugs.

The following scenarios may illustrate how a teen may be enticed to abuse a prescription drug. For example, a first sibling may overhear her parents discussing how a second sibling's ADHD (attention deficit hypertension disorder) medicine was making him less hungry. Because the first sibling is worried about her weight, she may start sneaking in and taking one of her second sibling's pills every few days. The second sibling may not find out that the medicine is being consumed faster than normal if the first sibling is careful in obtaining small, select doses every few days. As another example, a child may find an old bottle of painkillers that had been left over from his parents operation. The child may decide to try the painkillers for a variety of reasons. Because a doctor had prescribed the pills to the parents, the child may figure that meant they'd be fine to try or experiment with. Consequently, some people experiment with prescription drugs because they think they will help them have more fun, lose weight, fit in, and even study more effectively. Once again, these teenage views of prescription drugs are very alarming.

Of course, parents are crucial in helping prevent this behavior, but parents are largely unaware and feel ill-equipped to respond. Many households have a drawer filled with old prescription bottles containing leftover drugs. To reduce the availability of potentially addictive prescription drugs to teens, adults should secure such medications in a locked cabinet and dispose of any unused pills properly. However, these recommendations are often not effective because parents are forgetful of disposing of pills and locked cabinets can still be accessed by teenagers. Also, because prescription drugs have medical uses, teens often believe they are a safe alternative to street drugs and prescription drugs can be easier to get than street drugs. Family members or friends could have a prescription. In fact, some people think that prescription drugs are safer and less addictive than street drugs. After all, these are drugs that moms, dads, and even kid brothers and sisters use.

However, it is very dangerous to abuse prescription-drugs. Whether drug abusers are using street drugs or medications, drug abusers often have trouble at school, at home, with friends, or with the law. The likelihood that someone will commit a crime, be a victim of a crime, or have an accident is higher when that person is abusing drugs, no matter whether those drugs are medications/prescription drugs or street drugs.

Additionally, like all drug abuse, using prescription drugs for the wrong reasons has serious risks for a person's health. This risk is higher when prescription drugs like opioids are taken with other substances like alcohol, antihistamines, and anti-depressants. CNS depressants have risks, too. Abruptly stopping or reducing them too quickly can lead to seizures. Taking CNS (central nervous system) depressants with other medications, such as prescription painkillers, some over-the-counter cold and allergy medications, or alcohol can slow a person's heartbeat and breathing, and even kill. Abusing stimulants (like some ADHD drugs) may cause heart failure or seizures. These risks are increased when stimulants are mixed with other medicines, even ones like certain cold medicines. Taking too much of a stimulant can lead a person to develop a dangerously high body temperature or an irregular heartbeat. As a result, abusing prescription drugs can have many undesired and serious side effects that can spark long-lasting health problems in abusers.

Moreover, prescription drug addiction has been the most underreported drug abuse problem in the nation (National Institute of Drug Abuse). Addiction to and withdrawal from prescription drugs can be more dangerous than other substances because of the insidious nature of these drugs. Two types of the most commonly abused drugs are opioids and benzodiazepines. Opioids are generally used to control pain. Benzodiazepines, or tranquilizers, are used to manage anxiety. These drugs are prescribed for short-term use such as acute pain and anxiety that is in reaction to a specific event. They may also be prescribed for chronic pain or generalized anxiety. These drugs can be legally obtained from a doctor's prescription and recommendation.

However, taking prescription drugs in a way that hasn't been recommended by a doctor can be more dangerous than people think. In fact, its drug abuse and it's just as illegal as taking street drugs. On the flip side, prescription drugs are only safe for the individuals who who actually have prescriptions for them. That's because a doctor has examined these people and prescribed the right dose of medication for a specific medical condition. The doctor has also told them exactly how they should take the medicine, including things to avoid while taking the drug, such as drinking alcohol, smoking, or taking other medications. They also are aware of potentially dangerous side effects and can monitor patients closely for these.

Consequently, we cannot eliminate prescription drugs from our society since they do have beneficial effects if used properly. However, they are too easily accessible by people who have not been prescribed such prescription drugs. Thus, some new and novel solutions need to be presented that permit only valid/authorized/permitted users to access their medication containers/vials/receptacles without worrying that such medication containers/vials/receptacles are tampered with by unauthorized individuals.

### SUMMARY

The present disclosure provides a sealing apparatus configured for use with a receptacle, the sealing apparatus including an orifice for receiving one or more first breath samples from a user; an analyzing module for analyzing the one or more first breath samples received from the user via the orifice; a storage unit for storing the one or more first breath samples; and a fastening unit for securedly fixing the sealing apparatus to the receptacle once the one or more first breath samples have been stored in the storage unit.

The present disclosure also provides a method for affixing a sealing apparatus to a receptacle, the method including receiving one or more first breath samples from a user via an orifice; analyzing the one or more first breath samples received from the user via an analyzing module; storing the one or more first breath samples via a storage unit; and securedly fixing the sealing apparatus to the receptacle via a fastening unit once the one or more first breath samples have been stored in the storage unit.

The present disclosure also provides a method for manufacturing a sealing apparatus configured for use with a receptacle, the method including forming an orifice for receiving one or more first breath samples from a user; constructing an analyzing module for analyzing the one or more first breath samples received from the user via the orifice; constructing a storage unit for storing the one or more first breath samples; and forming a fastening unit for securedly fixing the sealing apparatus to the receptacle once the one or more first breath samples have been stored in the storage unit.

The present disclosure also provides a sealing apparatus, including an inlet for receiving a first breath sample from a user for fastening the sealing apparatus to an external device, the first breath sample including a plurality of organic compounds or molecules, a select combination of the plurality of organic compounds or molecules being used to create a unique breath profile; and a module for analyzing and storing the first breath sample as the unique breath profile; wherein the inlet further receives a second breath sample and compares the second breath sample with the first breath sample to determine whether access to the external device is permitted.

Further scope of applicability of the present disclosure will become apparent from the detailed description given hereinafter. However, it should be understood that the detailed description and specific examples, while indicating preferred embodiments of the present disclosure, are given by way of illustration only, since various changes and modifications within the spirit and scope of the invention will become apparent to those skilled in the art from this detailed description.

### BRIEF DESCRIPTION OF THE DRAWINGS

Various embodiments of the present disclosure will be described herein below with reference to the figures wherein:

FIG. 1 is a perspective view of a tamper-resistant system including a receptacle attached to a sealing apparatus having breath analysis capabilities, in accordance with the present disclosure;

FIG. 2 is a block diagram of breath analysis capabilities embedded within the sealing apparatus of the tamper-resistant system, in accordance with the present disclosure;

FIG. 3A is a perspective view of an exemplary locking mechanism for securedly fixing the receptacle to the sealing member, where the locking mechanism is in a locked state, in accordance with the present disclosure;

FIG. 3B is a perspective view of an exemplary locking mechanism for securedly fixing the receptacle to the sealing member, where the locking mechanism is in an unlocked state, in accordance with the present disclosure;

FIG. 4 is a flowchart illustrating an initial setup of inputting breath samples to enable the locking mechanism to securedly fix the receptacle to the sealing apparatus, in accordance with the present disclosure; and

FIG. 5 is a flowchart illustrating accessing the contents of the receptacle after an initial breath sample has been inputted and saved, in accordance with the present disclosure.

### DETAILED DESCRIPTION

Unless otherwise indicated, all numbers expressing quantities and conditions, and so forth used in the specification and claims are to be understood as being modified in all instances by the term “about.” In this application, the use of the singular includes the plural unless specifically stated otherwise. In this application, the use of “or” means “and/or” unless stated otherwise. Furthermore, the use of the term “including,” as well as other forms, such as “includes” and “included,” is not limiting. Also, terms such as “element” or “component” encompass both elements and components comprising one unit and elements and components that comprise more than one subunit unless specifically stated otherwise. The term “coupled to” means to be attached or connect to directly or indirectly or to be incorporated within.

Prior to describing the present disclosure in further detail, it will first be helpful to define various terms that will be used throughout the following discussion. For example:

As used in this description and in the appended claims, the word “container” does not necessarily refer to a rigid or a somewhat deformable structure, such as a “bottle,” “bottle portion,” or “bottle half” for containing liquid or any other substances. Rather, the word “container” in the present disclosure and in the appended claims can also mean a “box,” “packet,” “bag,” “portion of a bag,” “pocket of a bag,” or any such deformable/non-deformable structure for containing liquid and/or prescription drugs, such as pills/tablets/capsules or powders.

The term “container” may refer to a receptacle. The receptacle may be a container for disposal or storage. The term “container” may also include a bag, bin, bottle, bowl, box, can, canister, canteen, capsule, carton, casket, chamber, crate, flask, jar, jug, packet, package, pouch, receptacle, repository, sack, tank, tub, vase, vessel and/or vial. The “container” may be flexible or non-flexible (rigid) or semi-rigid. The “container” may be transparent, semi-transparent or non-transparent. The “container” may include any number of different substances (liquid or non-liquid or powder) or products or data or information.

The term “module” may refer to a self-contained component (unit or item) that is used in combination with other components and/or a separate and distinct unit of hardware or software that may be used as a component in a system, such as an analyzing system, including a tamper-resistant receptacle connected to a sealing apparatus. The term “module” may also refer to a self-contained assembly of electronic components and circuitry, such as a stage in a computer that is installed as a unit.

The term “analyze” may refer to determining the elements or essential features or functions or processes of a plurality of analyzing modules in a tamper-resistant device and/or to subject the plurality of analyzing modules in a tamper-resistant device to computational processing. The term “analyze” may further refer to tracking data and/or collecting data and/or manipulating data and/or examining data and/or updating data and/or inspecting data and/or distinguishing data on a real-time basis in an automatic manner and/or a selective manner and/or manual manner.

The term “storage unit” may refer to data storage. “Data storage” can refer to any article or material (e.g., a hard disk) from which information is capable of being reproduced, with or without the aid of any other article or device. “Data storage” can refer to the holding of data in an electromagnetic form for access by a computer processor. Primary storage is data in random access memory (RAM) and other “built-in” devices. Secondary storage is data on hard disk, tapes, and other external devices. “Data storage” can also refer to the permanent holding place for digital data, until purposely erased. “Storage” implies a repository that retains its content without power. “Storage” mostly means magnetic disks, magnetic tapes and optical discs (CD, DVD, etc.). “Storage” may also refer to non-volatile memory chips such as flash, Read-Only memory (ROM) and/or Electrically Erasable Programmable Read-Only Memory (EEPROM).

The term “computing subsystem” may refer to any type of programmable machine, such as a computer, where the programmable machine can execute a programmed list of instructions and respond to new instructions that it is given. The term “computing subsystem” may also refer to a machine for performing calculations automatically or to a machine that machine that manipulates data according to a list of instructions or to a programmable device that performs mathematical calculations and logical operations, especially one that can process, store and retrieve large amounts of data very quickly. The term “computing subsystem” may also refer to any type of device that stores and processes information, where the information is stored internally or externally either temporarily or permanently.

The term “fasten” or “fastening” may refer to adhere, affix, anchor, attach, band, bind, bolt, bond, brace, button, cohere, connect, couple, embed, establish, fix, grip, hold, hook, implant, link, lock, lodge, screw, seal, rivet, tack on, tighten, or unite. The term “fasten” or “fastening” may refer to linking/connecting/attaching/locking any type of materials in a removable/detachable/interchangeable manner.

There are many types of access prevention systems and methods that may be envisioned for preventing unauthorized users from accessing a product/device/apparatus. However, the prior art does not provide for any type of breath analysis systems used as access prevention systems and/or authorization systems. To this point, breath analysis has been used solely for the detection of diseases and to methods of detecting, collecting, and inspecting breath samples for specific trace compounds.

The potential for the use of exhaled breath as a diagnostic tool has long been recognized. Researchers have shown that the chemical composition of expired breath can be an accurate, timely, and painless indicator of the health of an individual. For example, researchers have used light absorption and emission by molecules as a means for qualitatively identifying which molecules are present in a mixture, and quantitatively determining what concentration of each is present. Commonly, molecules with two or more atoms show distinct absorptions in the infrared region of the spectrum. The detailed characteristics of these absorptions can be extremely sharp at low pressure for molecules that are in the gas phase, phase, enabling accurately determining organic compounds/molecules present in breath. Thus, breath analysis has been very limited to only diagnosing diseases by detecting molecules in alveolar breath only (further described below).

Consequently, there is no system or method for using breath analysis to allow or prevent (authorize) access to a product by using breath as an authorization variable (breath actuated prevention/authorization system). In particular, presently, there are no breath analysis systems or methods that prevent unauthorized users from accessing medication containers/vials/receptacles including prescription drugs. Also, what is desired is an optimized sample collection system with superior detection capabilities where the sample collection system and the detection system are small in size, ideally hand-held or portable, without compromising sensitivity and selectivity of the compound of interest for detection.

The present disclosure is intended to teach and/or suggest a system and method that prevent unauthorized users from accessing medication containers/vials/receptacles including, but not limited to, prescription drugs. In particular, the present disclosure relates to a tamper-resistant medical container/vial/receptacle that is connected to a sealing apparatus that includes breath detection and breath analysis capabilities. The present disclosure further relates to a method of manufacturing a tamper-resistant medical container/vial/receptacle that is securely fixed to a sealing apparatus having breath detection and breath analysis capabilities. The present disclosure further relates to providing a means for detecting and quantifying one or more compounds of interest in the exhaled breath from a collected sample, storing such unique breath sample, and comparing any other input breaths to the stored unique breath sample for determining whether to permit or deny access to the prescription drugs or any other contents within the receptacle (e.g., pills, powder, liquid, data, information).

The present disclosure further discloses a chemical analysis method related to human breath. The present disclosure further teaches a method of collecting human breath samples, analyzing such human breath samples, storing such human breath samples, and utilizing such human breath samples to permit or deny access to a receptacle containing any type of content. The breath profile/breath concentration profile can contain a number of different molecules and/or organic compounds detected in human breath (either dead air space breath or alveolar breath or a combination of both). For instance, human and animal breath contains hundreds of different trace volatile organic compounds (VOCs), in addition to the usual large amounts of H₂O and CO₂. Thus, these types of VOCs would be valuable in creating a unique breath profile for a user and using that unique profile for future comparison purposes to permit or deny access to contents of a receptacle.

Moreover, as mentioned above, there are two types of breath, that is, dead air space breath and alveolar breath. Dead air space breath is exhaled breath, whereas alveolar breath is breath located in the lungs. Dead air space breath may include hundreds, if not thousands, of different molecules/organic compounds. Also, alveolar breath, may include hundreds, if not thousands, of different molecules/organic compounds.

However, few molecules/organic compounds in each type of breath are common to all individuals. In fact, the majority of molecules/organic compounds in each type of breath is unique and is contained in varying numbers and concentrations per individual. As a result, breath can provide a unique snapshot/fingerprint/blueprint/signature of an individual, thus identifying/distinguishing such individual, as does DNA (Deoxyribonucleic acid), for example. In particular, the main role of DNA molecules is the long-term storage of information. DNA is often compared to a set of blueprints or a recipe, or a code, since it contains the instructions needed to construct other components of cells, such as proteins and RNA (Ribonucleic acid) RNA (Ribonucleic acid) molecules. The DNA segments that carry this genetic information are called genes, but other DNA sequences have structural purposes, or are involved in regulating the use of this genetic information. Similarly, breath stores/includes/encompasses information regarding an individual that is unique to that individual. By selectively counting unique molecules and/or organic compounds, and identifying such molecules and/or organic compounds, and measuring the concentration of each molecule and/or organic compound, a unique breath profile may be created that uniquely identifies or classifies or distinguishes or establishes or singles out a person and/or is used to actuate a prevention/authorization system.

As a result, in the exemplary embodiments of the present disclosure, breath is used as a blueprint or fingerprint or signature to identify/distinguish the unique breath of the authorized user of the vial/container/receptacle containing the prescription drugs. In other words, in the exemplary embodiments, breath can be employed to actuate the locking mechanism used to lock/affix/attach the receptacle to the sealing apparatus/member. In the exemplary embodiments, breath is used as a reliable actuation means since breath is a complex gas that includes hundreds, if not thousands, of different organic compounds/molecules, many, if not most, of which are unique to that individual. The rich assortment of chemical substances present in an individual's breath can reveal a great deal about the person doing the breathing, including providing a unique snapshot or blueprint or signature of the organic compounds/molecules (number and concentration) exuded from one's breath.

Therefore, analysis of breath samples for diagnostic purposes or non-diagnostic purposes has the advantage that the breath sample to be analyzed is collected from the patient in a non-invasive manner with a minimum of discomfort or inconvenience. In general, the trend in medicine is toward more non-invasive testing. Since breath is the only biological fluid fluid that may be obtained noninvasively and on demand, it is currently the matrix of choice for a number of applications, well beyond prescription drug containers.

However, breath samples need not be analyzed only for diagnostic purposes, as illustrated in conventional systems. In fact, each breath profile/chemical analysis profile/breath concentration profile of the exemplary embodiments is not characteristic of one or more medical conditions. In other words, the exemplary embodiments are not used for facilitating in the diagnosis of medical conditions. The breath concentration profile/spectroscopic breath analysis profile is used as a blueprint or fingerprint or signature of the organic compounds/molecules located in the breath of the user.

In the exemplary embodiments, the obtained breath profile is not compared to a generic breath profile stored in a database. The breath profile is compared against a breath profile previously stored in the sealing apparatus itself by a specific and unique user. In conventional breath diagnostic systems, a user's breath profile is compared against a generic breath profile that indicates a specific disease. In contrast, the exemplary embodiments of the present disclosure do not pertain to breath profiles that diagnose diseases. In fact, there are no generic breath profiles. The breath profiles of the exemplary embodiments are unique to a user since they are derived from one user, not a group of users displaying the same characteristics (such as a particular disease). There is no database of generic breath profiles that indicate a disease. The memory device of the present disclosure only stores the breath profile of a specific user, which is unique to that user. Once the user inputs a unique breath sample, that unique breath sample is stored locally, in the sealing apparatus, where the sealing apparatus then immediately and automatically goes into a locked mode. In other words, the sealing apparatus is locked into the external device (e.g., a medication container or vial including prescription drugs) once a valid breath sample has been obtained and saved.

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

Embodiments will be described below while referencing the accompanying figures. The accompanying figures are merely examples and are not intended to limit the scope of the present disclosure.

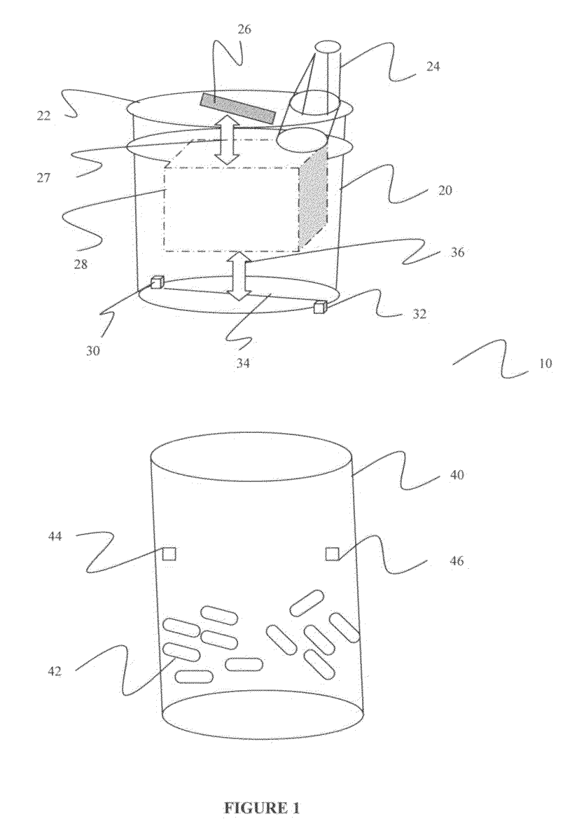

With reference to FIG. 1, there is presented a perspective view of a tamper-resistant system including a receptacle attached to a sealing apparatus having breath analysis capabilities, in accordance with the present disclosure.

The tamper-resistant system 10 of FIG. 1 includes a sealing apparatus 20 and a receptacle 40. The sealing apparatus 20 includes a top surface 22 of the sealing member 20, an orifice 24, an indicator display 26, an analyzing module 28, a first connector 30, a second connector 32, a locking mechanism 34, a first communication means 36, and a second communication means 27. The receptacle 40 includes prescription drugs 42, a first recess 44, and a second recess 46.

In operation, the detecting and collecting of organic compounds/molecules in a breath sample (such as a human breath sample, animal breath sample and/or plant breath sample), may include a person breathing or exhaling into an orifice 24 of a sealing apparatus 20 to absorb at least one breath molecule/organic compound. The breath is collected in an exhaled breath collector located in the analyzing module 28. The analyzing module 28 analyzes the breath sample and extracts one or more molecules and/or organic compounds. This data is stored in a storage means, such as a memory (RAM, ROM, EEPROM, SRAM, SDRAM, Flash memory, Holographic memory, etc.). The results of the analyzing module 28 may be used to trigger at least two other capabilities.

First, the analyzing module 28 may be used to trigger the indicator display 26 to display whether authorization/access is permitted via the second communication means 27. The indicator display 26 may be a visual indicator, an audible indicator, and/or a combination of a visual and audible indicator. The visual indicator may be a light emitting diode (LED) operating in a plurality of modes and colors. The indicator display 26 may be located on the top surface 22 of the sealing apparatus 20. However, it is envisioned that one skilled in the art may position the indicator display 26 on any portion of the sealing apparatus 20 or the receptacle 40 in accordance with design preferences. The indicator display 26 may display any type of information in any simple or complicated manner. For example, the words, “access permitted” and “access denied” may be displayed to indicate the result or the words “open” and “closed” may be displayed to indicate the result. However, any type of message conveying any type of information may be indicated on the indicator display 26. Also, the indicator display 26 may be of any size imaginable, from a few millimeters to a few inches and constructed from any type of materials (LEDs, LCDs (liquid crystal displays), or flexible displays). In addition, the indicator display 26 is optional.

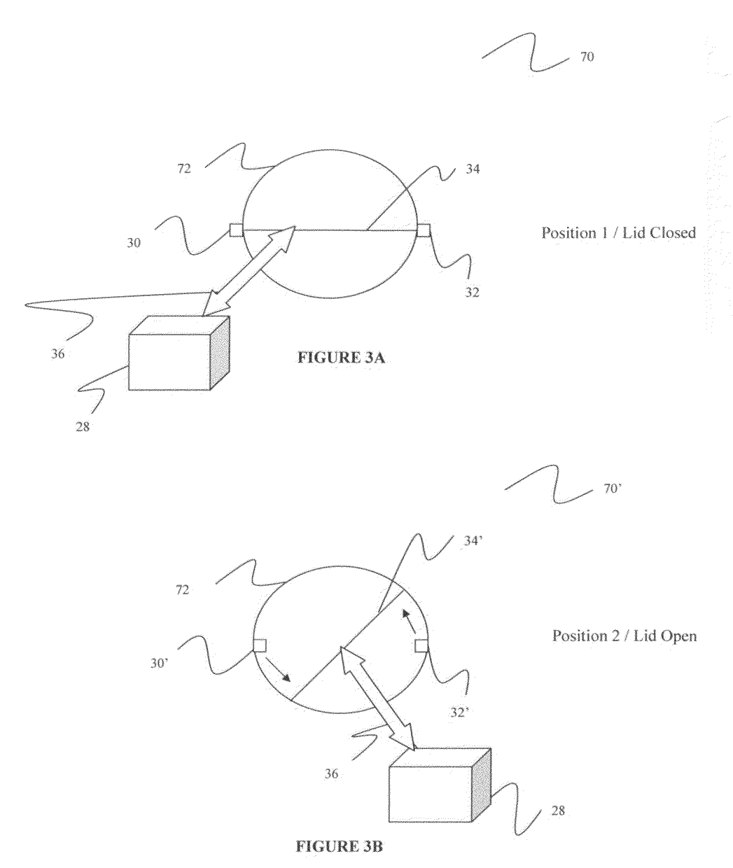

Second, the analyzing module 28 may be used to trigger the locking mechanism 34 via a first communication means 36. The locking mechanism 34 may be any type of electrical, mechanical or electromechanical mechanism used for securedly fixing the sealing apparatus 20 to the receptacle 40. As an exemplary illustration, the locking mechanism 34 is merely a merely a mechanical rod with a first connector 30 at one end and a second connector 32 at the other end. The locking mechanism 34 is linked to the analyzing module 28 via a first communication means 36. FIGS. 3A and 3B below further illustrate how such a simple locking mechanism 34 may be actuated based on a breath input via the orifice 24.

The receptacle 40 may be connected to the sealing apparatus 20 before or after a breath sample has been obtained via the orifice 24. In other words, a breath sample can be obtained before or after a locked state has been achieved. Optionally, a sensor may be positioned on either the receptacle 40 or the sealing apparatus 20 to detect whether a securedly fixed connection has been achieved (described further below).

The receptacle 40 includes prescription drugs 42. In addition, the receptacle 40 includes a first recess 44 and a second recess 46 for connecting the receptacle 40 to the sealing apparatus 20 via the first connector 30 and the second connector 32, respectively. However, this type of connection is merely exemplary. Any number of connections may be used by one skilled in the art to achieve a securedly fixed connection by electrical, mechanical or electro-mechanical means.

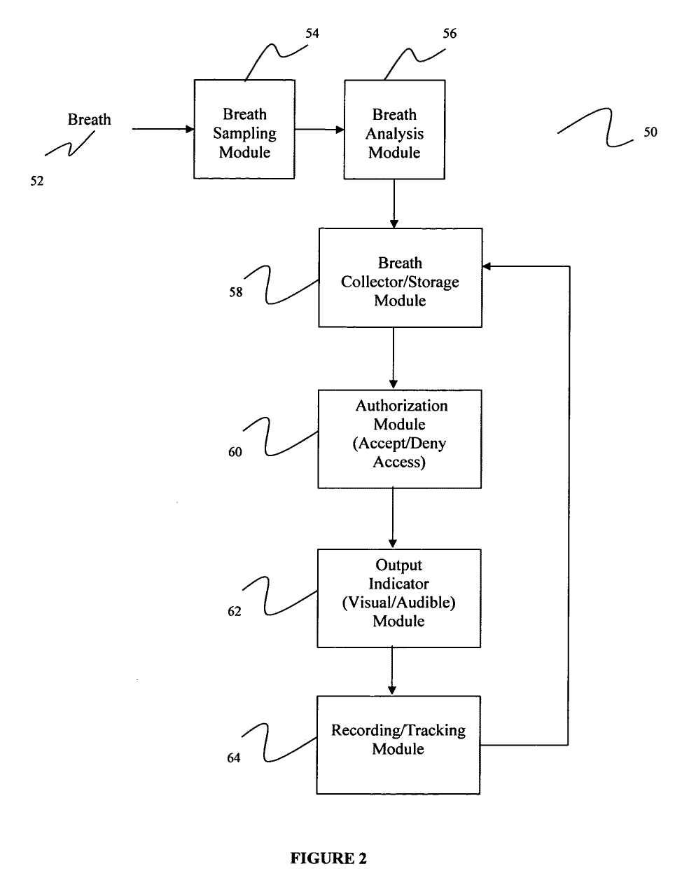

With reference to FIG. 2, there is presented a block diagram of breath analysis capabilities embedded within the sealing apparatus of the tamper-resistant system, in accordance with the present disclosure.

The block diagram 50 of FIG. 2 includes a breath 52, a breath sampling module 54, a breath analyzing module 56, a breath storage module 58, an authorization module 60, an output indicator module 62, and a recording/tracking module 64.

The breath sampling module 54 receives one or more breaths and takes one or more samples from that breath. The samples may be a section, a fragment, an instance, a part, a pattern, a piece, a portion, a segment or a unit of breath. Several samples may be extracted for accuracy and a portion of each sample may be used or a portion of select samples may be used. In other words, the breath sampling module 54 can receive several input breaths and selectively decide which breaths to accept. The breath sampling module 54 can accept one sample from one breath or a plurality of samples from several breath inputs from the same user.

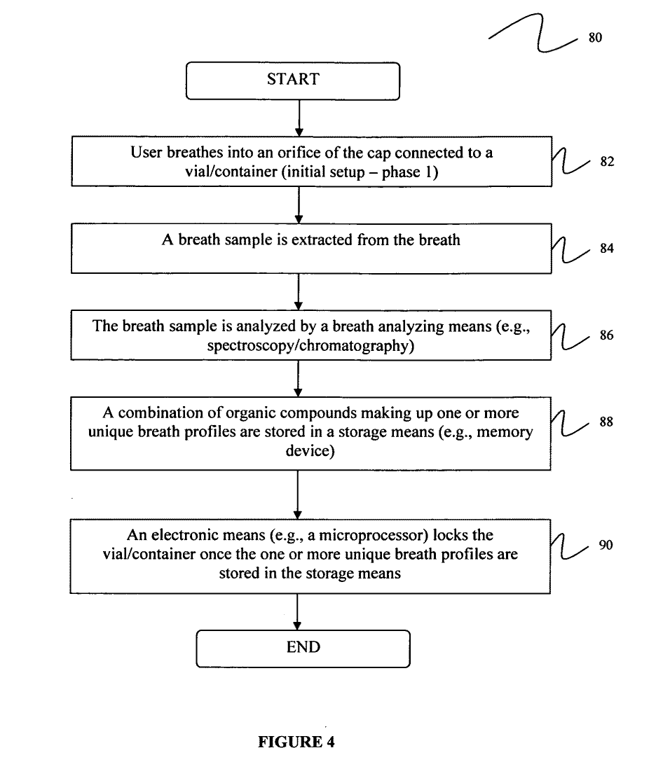

The breath analyzing module 56 analyzes the one or more samples of breath provided by the breath sampling module 54. The breath analysis can be executed by using any type of breath analysis techniques, such as spectroscopy/spectrometry and/or gas chromatography. One skilled in the art can envision any type of suitable breath analysis techniques, as described below with reference to FIG. 4.

Breath analysis refers to extracting a number of molecules and/or a number of organic compounds and/or a concentration for each of the molecules and/or organic compounds. The breath analyzing module 56 can be pre-programmed to selectively choose which of the plurality of molecules and/or organic compounds to use for creating a unique breath profile. The breath analyzing module 56 can choose select uncommon molecules and/or uncommon organic compounds to create a unique breath profile. The breath analyzing module 56 can identify the concentration of each select uncommon molecule and/or organic compound. The unique breath profile can be created by using a plurality of different variables contained in breath that would be deemed satisfactory to provide for a unique breath profile. The exemplary embodiments are not limited to any specific variables or to any specific specific concentration of variables. All these variables can be predetermined/preset (factory settings/default settings) or can be uniquely prepared/modified based on the user.

The breath analyzing module 56 can use/manipulate any type of electronic means/electronic devices to analyze the breath sample provided by the breath sampling module 54. For example, any type of microprocessor may be used to execute such operations.

The breath storage module 58 stores the selected plurality of molecules and/or plurality of organic compounds in a memory device to be processed by, for example, a microprocessor. The storage device may be located within the sealing apparatus 20. However, it is contemplated than an external storage device may be utilized in conjunction with an internal storage device, where the two storage devices communicate via wireless means. In other words, an external storage device may be located in a doctor's office, where the patient inputs a breath sample and that breath sample may be transferred from the external device to the internal storage device located in the sealing apparatus 20. The breath storage module 58 may include a storage device/memory device of any suitable size.

The authorization module 60 determines whether there is a match between a unique breath sample saved in the memory device and a second breath received via the orifice 24. In other words, several breaths from several different individuals may be entered via the orifice 24 and a determination needs to be made whether any of those subsequent breath samples matches the first breath sample (during initialization) in order to permit access to the contents of the receptacle 40. The authorization module 60 optionally communicates with the output indicator module 62 to provide for a means for informing the user whether access has been permitted or denied.

The output indicator module 62 can be a visual or an audible or a combination of a visual and audible indication means. The visual indicator may be a light emitting diode (LED) operating in a plurality of modes and colors. The indicator display 26 may be located on the top surface 22 of the sealing apparatus 20. However, it is envisioned that one skilled in the art may position the indicator display 26 on any portion of the sealing apparatus 20 or the receptacle 40 in accordance with design preferences. The indicator display 26 may display any type of information in any simple or complicated manner.

The recording/tracking module 64 can record and track the input activity. In other words, each time a breath sample is entered via the orifice 24, an instance of such input may be recorded and saved in the memory device. For example, if a certain unauthorized individual is constantly attempting to access the contents of the receptacle, the recording/tracking module 64 can save a unique breath sample of such individual and keep records of when such unauthorized individual made such attempts. Such information can be transmitted to a computing subsystem (such as a personal computer (PC) or other mobile devices, such as a cell phone), where the authorized user can track who is making attempts to access the contents of the receptacle 40. Additionally, the original breath profile of the authorized user may be monitored for slight deviations and the electronic processing means may decide to provide slight/minor updates to the existing original breath profile based on any deviations experienced (described further below). This may be executed in an automatic manner by the electronic processing means located in the breath analysis module 56.

With reference to FIG. 3A, there is presented a perspective view of an exemplary locking mechanism for securedly fixing the receptacle to the sealing member, where the locking mechanism is in a locked state, in accordance with the present disclosure.

The locking mechanism of FIG. 3A includes a first position of the sealing apparatus apparatus 70 and a bottom portion of the sealing apparatus 72. FIG. 3A shows similar elements to those of FIG. 1. In particular, the common elements are: the analyzing module 28, the first connector 30, the second connector 32, the locking mechanism 34, and the first communication means 36. All these similar elements are displayed in a “first position.”

The “first position” indicates that the sealing member 20 is locked into the receptacle 40. In other words, the sealing apparatus 20 is in a locked state. This is the state in which the locking mechanism enters into when a breath sample has been saved.

With reference to FIG. 3B, there is presented a perspective view of an exemplary locking mechanism for securedly fixing the receptacle to the sealing member, where the locking mechanism is in an unlocked state, in accordance with the present disclosure.

The locking mechanism 70′ of FIG. 3B includes a second position of the sealing apparatus 70′ and a bottom portion of the sealing apparatus 72. FIG. 3B shows similar elements to those of FIG. 1. In particular, the common elements are: the analyzing module 28, the first connector 30′, the second connector 32′, the locking mechanism 34′, and the first communication means 36. All these similar elements are displayed in a “second position.” Some of the elements are designated with a “prime” in order to indicate that they have shifted to the “second position.”

The “second position” indicates that the sealing member 20 is not locked into the receptacle 40. In other words, the sealing apparatus 20 is in an unlocked state. This is the state in which the locking mechanism confirms that a valid breath has been entered and allows access to the contents of the receptacle.

In summary, when a breath sample has been verified as valid, the first connector 30 and the second connector 32 are moved from a first position 70 to a second position 70′, which is a depressed position, so that the first connector 30′ and the second connector 32′ are located within the bottom portion of the sealing apparatus 72. In addition, the locking mechanism 34 moves from a first position to a second position, where the locking mechanism 34′ is shifted in either direction away from the first connector 30 and the second connector 32. In all positions, the analyzing module 28 is connected to the locking mechanism 34 via the first communication means 36. In this exemplary embodiment, an electrical component (elements 28, 36) actuates a mechanical component (elements 30, 32, 34) in order to achieve a locking/unlocking mechanism. In other words, this is an electromechanical locking system. However, any type of locking mechanism is envisioned by one skilled in the art.

With reference to FIG. 4, there is presented a flowchart illustrating an initial setup of inputting breath samples to enable the locking mechanism to securedly fix the receptacle to the sealing apparatus, in accordance with the present disclosure.

The initial setup flowchart 80 includes the following steps. In step 82, a user breathes into an orifice of the cap connected to a vial/container. This is the initial setup or phase one of the process. In step 84, a breath sample is extracted from the breath. In step 86, the breath sample is analyzed by a breath analyzing means (e.g., spectroscopy and/or chromatography). In step 88, a combination of organic compounds/molecules making up one or more unique breath profiles are stored in a storage means (e.g., memory device). In step 90, an electronic means (e.g., a microprocessor) locks the vial/container once the one or more unique breath profiles are stored in the storage means.

Spectroscopy pertains to the dispersion of an object's light into its component colors (i.e. energies). By performing this dissection and analysis of an object's light, researchers can infer the physical properties of that object (such as temperature, mass, luminosity, number of molecules, number of organic compounds, and composition or concentration of molecules/organic compounds). Spectrometry is the spectroscopic technique used to assess the concentration or amount of a given species. In those cases, the instrument that performs such measurements is a spectrometer or spectrograph. Spectroscopy and/or spectrometry is often used in physical and analytical chemistry for the identification of substances through the spectrum emitted from or absorbed by them. Mass spectroscopy is a detection method, which can be coupled with chromatography or sample directly from the headspace of a sample, which ionizes, fragments, and rearranges a molecule under a given set of conditions and makes identification of the molecular weight/charge (m/z) of molecules possible. A mass spectrum is a plot showing the mass/charge ratio versus abundance data for ions from the sample molecule and its fragments.

In addition, any type of spectrometry and/or spectroscopy can be used, such as, but not limited to, electromagnetic spectroscopy, electron spectroscopy, mass spectroscopy, absoprtion spcetroscopy, emission spectroscopy, infrared spectroscopy, ultraviolet spectroscopy, thermal spectroscopy, laser spectroscopy, and/or scattering spectroscopy. One skilled in the art can envision any type of spectroscopy and/or spectrometry techniques used to analyze breath from breath samples.

Chromatography pertains to a broad range of physical methods used to separate and or to analyze complex mixtures. The components to be separated are distributed between two phases: a stationary phase bed and a mobile phase which percolates through the stationary bed. Chromatography is a collective term for a family of laboratory techniques for the separation of mixtures. It involves passing a mixture dissolved in a “mobile phase” through a stationary phase, which separates the analyte to be measured from other molecules in the mixture and in the mixture and allows it to be isolated. Chromatography is the physical separation of two or more compounds based on their differential distribution between two phases, the mobile phase and stationary phase. The mobile phase is a carrier gas that moves a vaporized sample through a column coated with a stationary phase where separation takes place. When a separated sample component elutes from the column, a detector, such as a Flame Ionization Detector (FID) or an Electrochemical Detector (ECD), converts the column eluent to an electrical signal that is measured and recorded.

Any type of chromatography may be used, including, but not limited to, gas chromatography, affinity chromatography, and/or ion exchange chromatography. One skilled in the art can envision any type of chromatography techniques used to analyze breath from breath samples.

Additionally, the present disclosure is not limited to only these two types of breath analysis techniques. Any type of breath analysis technique or combination thereof may be used to analyze breath samples from humans, animals, and/or plants. For example, any type of chemical sensor may be used. In addition, several vapor sensing technologies, including conducting polymers, electrochemical cells, infrared spectroscopy, ion mobility spectrometry, metal oxide semiconductor, photo-ionized detectors, Fourier transforms, non-dispersive infrared spectrometry, elected ion flow tubes, and surface acoustic wave sensors, have been evaluated for detection of compounds in the breath. Sensor sensitivity, selectivity, operating life, shelf-life, drift, linearity, initial cost, recurring costs, warm-up time, analysis time, power consumption, portability and calibration needs may also be evaluated to decide on the desired technique.

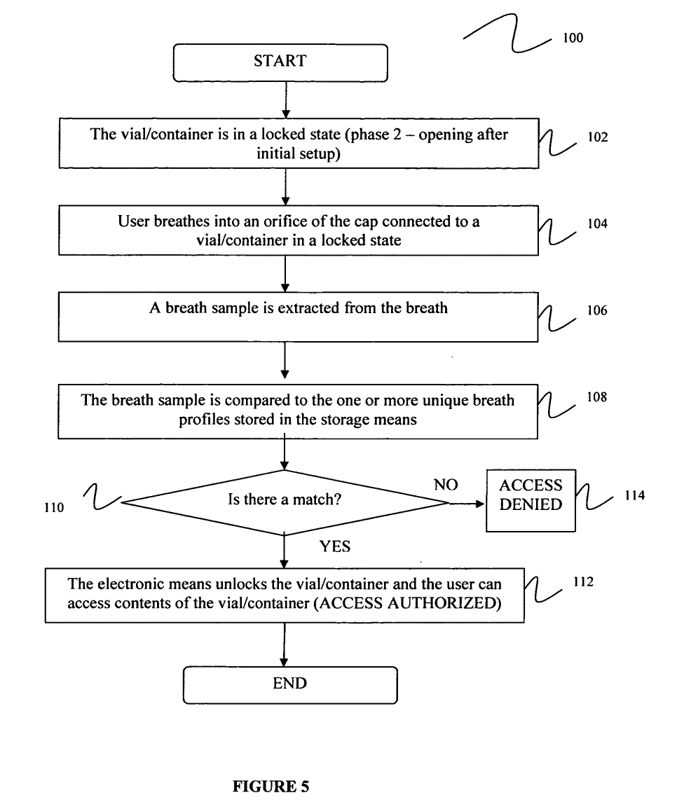

With reference to FIG. 5, there is presented a flowchart illustrating accessing the contents of the receptacle after an initial breath sample has been inputted and saved, in accordance with the present disclosure.

The authorization/access flowchart 100 includes the following steps. In step 102, the vial/container is in a locked state. This is the second phase or opening after initial setup, also referred to as access/authorization phase. In step 104, the user breathes into an orifice of the cap connected to a vial/container in a locked state. In step 106, a breath sample is extracted from the breath. In step 108, the breath sample is compared to the one or more unique breath profiles stored in the storage means. In step 110, a decision is made whether there is a match between the saved breath profile and the recently inputted breath sample. If there is no match, then the process flows to step 114 where access is denied. This simply means that the locking mechanism remains in a locked state and the user attempting to access the prescription drugs is denied from access. If there is a match, the process flows to step 112, where the electronic means unlocks the vial/container and the user can access contents of the vial/container (access is authorized). This simply means that the user attempting to access the prescription drugs is permitted to do so because a match has been confirmed. The process then ends.

A match can occur in a variety of ways. For example, a plurality of molecules may be collected or a plurality of organic compounds may be collected or a combination of a plurality of molecules and organic compounds may be collected. This plurality of molecules and/or organic compounds may be extracted from dead air space breath, from alveolar breath or a combination of dead air space breath and alveolar breath. The match can require any number and concentration of matching molecules and/or organic compounds.

For example, it is preferable that uncommon molecules and/or organic compounds be selected for comparison. There may be hundreds or even thousands of molecules and/or organic compounds from which to select a number of desirable combinations for comparison purposes. For example, 100 uncommon organic compounds may be selected to form a unique breath profile for a person. However, 200, 300 or even 400 uncommon organic compounds may be selected to form a unique breath profile for a person. In addition, 200 unique molecules and 200 unique organic compounds may be selected to form a unique breath profile for a person. In other words, any uncommon/common organic compounds may be selected, any uncommon/common molecules may be selected, or any combination of molecules and organic compounds may be selected to form any type of desirable unique breath profile (number of items and concentration of items may be examined). Of course, any type of variables may be measured and/or identified and/or collected that would be satisfactory for creating any type of unique breath profile pertaining to a person. One skilled in the art could envision using any chemical analysis techniques and using any chemical variables with any type of chemical characteristics to obtain a preferred unique breath profile.

Optionally, the sealing apparatus and receptacle may be one unit. In the exemplary embodiments, the sealing apparatus and receptacle are two separate units. However, such devices may be combined into one unit (uniform and continuous) based on user, manufacturing, and design preferences.

Optionally, a sensing device may be included in the tamper-resistant system to detect when the sealing member has been inserted into/attached to an external device (such as, but not exclusively, a receptacle having a plurality of prescription pills). The sensing device may be included within the sealing member or within the receptacle or a portion of the sensing device may be included in both the sealing member and the receptacle. The sensing device device may be included on an inner surface of the receptacle/sealing member or may be included on an outer surface of the receptacle/sealing member. The sensing device can be any type of sensor that measures a physical quantity and converts it into a signal which can be read by an observer or by an instrument. The sensor(s) may be manufactured on a microscopic scale as micro-sensors using MEMS (micro-electromechanical systems) technology.

Optionally, more than one orifice may be used. For example, two orifices may be used on the sealing apparatus. One orifice may pertain to a first person and one orifice may pertain to a second person. One person may be the husband and the other person may be the wife who is sharing the same type of medication. Again, one person may be the husband using one type of medication and the other person may be the wife used a different type of medication, where the receptacle includes two chambers, one for each type of medication. In other words, the receptacle can be a multi-chambered receptacle, where each chamber includes different contents, and each of the contents can be accessed by a separate orifice. Also, each of the contents can be connected to one orifice and different breath profiles can trigger different chambers having different contents. One person may be a patient and one person may be a nurse/home-care assistant that is authorized to access the medication for providing such medication to the patient. One person may be a child and one person may be a parent that has authority to access the prescription drugs and make sure they are not abused by the child. The possibilities are limitless.

Optionally, there may be one orifice that accepts more than one unique profile. In other words, 2 or 3 breath profiles of 2 or 3 different individuals may be programmed into the memory. All the users may use the same orifice or separate orifices may be provided for each user breath profile. More than one unique breath profile may be permissible for situations where a nurse/doctor/parent/other health care professional needs to access the receptacle. Of course, each orifice can trigger a different breath profile or in the alternative different breath profiles can be triggered by using the same orifice, as long as the memory device has the different breath profiles stored and indentified to specific users.

Optionally, any type of sealing mechanism may be used to securely fix the receptacle to the sealing apparatus. The sealing mechanism may be mechanical, electrical, or a combination of an electromechanical mechanism. Several different locking mechanisms may be envisioned by one skilled in the art.

Optionally, any type of timing requirements may be programmed and saved into the tamper-resistant system. For example, once the receptacle has been accessed, a timing requirement may be in place that does not allow access (even if the breath profiles match) within the next 2 hours, 4 hours, 6 hours, etc. In fact, even daily timing requirements may be enabled. This system would prevent the authorized user of the prescription drugs to abuse his/her own medication. For example, if a user accesses the medication at 10:00 am, and a 4 hour timing requirement is in place, the user cannot access the medication again at 11:00 am (even if a match if confirmed). The user would have to wait until the timing requirement expires (e.g., 2 pm in this instance).

In the exemplary embodiments, the breath profile can be loaded directly into the sealing member that is attached to the receptacle. Optionally, the breath profile can be loaded by a patient from an external location. Such external location could be a doctor's office or a pharmacy that provides for such sealing members. Once the breath profile is loaded in the sealing member in the external location it can be transferred (say by wireless means) to the memory device located in the sealing member by a doctor or nurse or the patient/user.

Optionally, data from a particular patient may be stored so that multiple samples over an extended period of time may be taken. This permits a baseline to be established for a particular patient/user, and trend analysis is performed on the resulting data, relative to the database of breath profiles. If there is an acute and significant change in the chronic condition of the patient's breath, indications of this change may be communicated to a physician or healthcare provider or to the patient and may be used to adjust or modify the breath profiles for access purposes. In other words, this is a dynamic system that allows for updated/revised breath profiles to be stored based on a user's changed circumstances. For example, a user may take different medications that later affect his breath profile. If such is the case, the user can modify the breath profile to account for such changed conditions.

Optionally, the sealing apparatus may include a reset button that allows a user to reset his/her breath profile. However, the reset function would not be activated until an initial setup has been established. In other words, a user must first enter a first breath profile into the system, that breath profile must be saved into the memory device, and the sealing apparatus must be locked to a receptacle. Once a lock has been confirmed, the user must first verify that he/she is the authorized user and then would be able to reset the breath profile.

Optionally, the results obtained from quantitative or qualitative analyses, the minimum detection analyses, and the minimum/maximum range analyses can be stored in a memory device and examined. Depending on the desired information, a computing means, such as a microprocessor, may check for significant changes in the quantitative or qualitative analyses for selected components, over time for a particular patient/user as compared to previous breath profiles. In other words, this is a dynamic system that allows the patient/user to constantly provide updated breath profiles and the system to adjust/modify/reconfigure the settings to provide for the most accurate, exact, concrete, distinct or definite variables and variable concentrations for creating a reliable breath match.

Optionally, the breath analyzer may request a new breath sample every few predetermined periods of time. For example, depending on the medication, the breath analyzer may request a breath sample every week, every 2 weeks, every month, etc. based on the type of medication, the timing of the doses, etc. These new breath samples may be compared to prior breath samples for accuracy and for any significant changes in the health of the patient/user. These new breath samples may also be compared to prior breath samples for updating/revising the new breath sample. As a result, breath samples may be updated/revised periodically. The breath analyzer may make such requests automatically in pre-established or pre-designated or preset time periods, for instance, based on a doctor's recommendations.

Additionally, the present disclosure is not limited to attaching such a sealing apparatus containing a breath analysis component/element/module only to a receptacle having a plurality of prescription pills. A medication bottle/container/receptacle is not the only connection to such a sealing member. The sealing member can attach to anything that requires a secure connection that one desires no tampering with. The sealing member can be modified to fit onto/into a computer system, a credit card, security devices, DVDs, CDs, portable devices, MP3 players, automobile access systems, anti-theft devices, key-ignition systems, machine-access systems, door systems, office systems, any type of appliances, etc. In other words, any type of electrical devices may be used in conjunction with the breath analysis sealing member of the present disclosure. Electrical devices refer to any devices/apparatuses/systems that produce or are powered by electricity and/or send and receive signals. There are an unlimited number of applications where such sealing apparatus can be modified in design to aid in the prevention of unauthorized access.

The tamper-resistant breath collection and analysis apparatus according to the exemplary embodiments also has several more advantages:

1. Portable: the tamper-resistant breath collection apparatus allows for collection of the breath sample in any environment.

2. User-friendly: the tamper-resistant breath collection apparatus is easy to operate and presents no significant resistance to sampling via inhalation and exhalation. In addition, the detection system processes the sample and can provide the desired response in a simple graphical user interface.

3. Disposable: the tamper-resistant breath collection apparatus provides no possible exposure to cross-contamination or exposure to infectious pathogens from another patient/doctor/nurse/medical professional/home attendant.

4. Efficient sampling: The tamper-resistant breath collection apparatus can control the breath sampling by collecting only the alveolar breath component, only the dead air space breath or a combination of the two types of breath.

5. Concentration of sample: The tamper-resistant breath collection apparatus may allow for the two types of breath to be sampled over multiple breaths, thus improving the possibility of detecting compounds of interest that are present at extremely low concentrations in the breath.

6. Prevention of unauthorized users: The tamper-resistant breath collection apparatus effectively disallows unauthorized users from accessing the contents of a receptacle by requiring a correct/accurate breath profile match. The tamper-resistant breath collection apparatus prevents unauthorized persons from abusing prescription drugs, as well as the authorized user from abusing his/her own prescription drugs.

Optionally, while threaded connections are utilized to connect various components in the described embodiments, many other forms of connections, such as snap together connections, twist-to-lock connections and the like also can be utilized. The present disclosure may also include a twist-on or snap-on spout or nozzle, preferably of a tapered conical or substantially cylindrical shape, and internally divided. The spout or nozzle may be adapted to be sealed by an end cap, a plug, by helically twisting the “overcap” upon a “scaling rod,” or by sliding upon an internal shaft affecting a seal when screwed or pushed downwards towards the bottle. Any type of electrical, mechanical or electromechanical connection may be used by one skilled in the art to connect a sealing member to a receptacle.

Optionally, the receptacle and sealing member of the present disclosure may be constructed of a clear or transparent or translucent material in order to better identify the content contained within the receptacle and/or the sealing member.

Additionally, all the receptacles of the present disclosure are not limited to any particular shape or design. Although the receptacles are described and depicted herein as being of generally cylindrical upstanding form, the configurations of the containers is a matter of design choice. The use of generally cylindrical containers is described because it gives the receptacle a readily acceptable appearance and shape, and because generally cylindrical container shapes tend to work well if one also desires to make use of generally cylindrical, externally threaded or snap-on container necks. Moreover, generally cylindrical containers tend to efficiently provide good fluid-carrying capacity at relatively low manufacturing cost. While opaque, single-thickness materials may be preferred for use, transparent or plural-layer materials may be used, if desired, to enhance visibility, to provide added insulating capability, or for other purposes.

Furthermore, all the receptacles of the present disclosure may attach to a plurality of different caps or lids or sealing members/apparatuses, and each of the one or more caps or lids or sealing members may have a strap connected to the body. All the receptacles of the present disclosure may include one or more cooling elements to cool the contents contained within the chambers or containers. All the receptacles and/or sealing apparatuses of the present disclosure may be of different widths and/or heights. All the receptacles of the present disclosure may have different caps of different shapes and/or sizes with a plurality of fastening means. All the receptacles and/or sealing apparatuses of the present disclosure may have interchangeable parts. Finally, all the receptacles and/or sealing members of the present disclosure may be constructed by any manufacturing means. For example, blow molding, injection molding technology may be utilized. A plurality of different types of thermoplastic resins may be utilized in any type of blow molding/injection molding techniques.

It will be understood that there are to be no limitations as to the dimensions and shape of the receptacle and/or sealing apparatus, including the storage compartment, or the materials from which the receptacle and/or sealing apparatus is manufactured or the electronics that may be used to run such a tamper-resistant system. The receptacle may be constructed to resemble any commercially available bottle/container for holding a liquid or non-liquid substance (e.g., pills, tablets, powder, personal items, information) and may be manufactured from any suitable plastic, glass or metal material. Furthermore, it should be understood that the receptacle of the present disclosure may be adapted to store any suitable liquid, such as, for example, liquid medications, liquid drugs, but also water, juice, milk, carbonated sodas, protein shakes, energy drinks, beer, wine, and liquor. Such a sealing apparatus can be used to prevent access by unauthorized individuals from any types of liquids. It is contemplated that such a removable/detachable sealing apparatus can be sold separately and attached to any type separately and attached to any type of device/apparatus/structure/bottle.

It will be appreciated that variations of the above-disclosed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

Accordingly, the present disclosure prevents the unauthorized access of prescription drugs (or any types of content) from a receptacle (vial/medical container), thus minimizing or even eliminating the risk that a teenager or other type of unauthorized user accesses the prescription drugs stored in the receptacle. The unauthorized access is prevented by providing a breath profile comparison means for permitting access only if a breath match has been confirmed.

Having described the invention above, various modifications of the techniques, procedures, material and equipment will be apparent to those in the art. It is intended that all such variations within the scope and spirit of the appended claims be embraced thereby.

The foregoing examples illustrate various aspects of the invention and practice of the methods of the invention. The examples are not intended to provide an exhaustive description of the many different embodiments of the invention. Thus, although the foregoing invention has been described in some detail by way of illustration and example for purposes of clarity and understanding, those of ordinary skill in the art will realize readily that many changes and modifications can be made thereto without departing form the spirit or scope of the invention.

What is claimed is:

1. A sealing apparatus configured for use with a receptacle, the sealing apparatus comprising:
an orifice for receiving one or more first breath samples from a user;
an analyzing module for analyzing the one or more first breath samples received from the user via the orifice;
a storage unit for storing the one or more first breath samples; and
a fastening unit for securedly fixing the sealing apparatus to the receptacle once the one or more first breath samples have been analyzed by the analyzing module and stored in the storage unit.

2. The sealing apparatus according to claim 1, wherein the receptacle is a medical container configured to hold prescription drugs and the one or more first breath samples indicate a chemical breath profile of the user.

3. The sealing apparatus according to claim 1, wherein after the fastening unit securedly fixes the sealing apparatus to the receptacle, the user is permitted to input one or more second breath samples via the orifice.

4. The sealing apparatus according to claim 3, further comprising a comparison module for comparing the one or more second breath samples to the one or more first breath samples to obtain a result;
wherein the comparison module compares a plurality of select organic compounds detected in the breath samples and a concentration of each of the plurality of select organic compounds detected in the breath samples with predetermined organic compounds and predetermined ranges of concentration of select organic compounds.

5. The sealing apparatus according to claim 4, wherein the result either maintains the fastening unit in a locked state or actuates the fastening unit to an unlocked state.

6. The sealing apparatus according to claim 5, further comprising an indicator unit for indicating whether the fastening unit is in the locked state or in the unlocked state;
wherein the indicator unit is one or more of the following: a visual indicator, an audible indicator, or a combination of the visual and the audible indicators.

7. The sealing apparatus according to claim 1, wherein the analyzing module is a chemical breath component analyzer configured to:
(i) count each of a plurality of select organic compounds from the one or more first breath samples received from the user via the orifice; and
(ii) determine a concentration of each of the plurality of the select organic compounds.

8. The sealing apparatus according to claim 7, wherein each of the plurality of select organic compounds is selected based on one or more of the following: predetermined uncommon organic compounds, detected uncommon organic compounds, and uncommon concentrations of organic compounds.

9. The sealing apparatus according to claim 7, wherein the plurality of select organic compounds are extracted from one or more of the following: dead air space breath, alveolar breath, or a combination of dead air space breath and alveolar breath.

10. A method for affixing a sealing apparatus to a receptacle, the method comprising:
receiving one or more first breath samples from a user via an orifice;
analyzing the one or more first breath samples received from the user via an analyzing module;
storing the one or more first breath samples via a storage unit; and
securedly fixing the sealing apparatus to the receptacle via a fastening unit once the one or more first breath samples have been analyzed by the analyzing module and stored in the storage unit.

11. The method according to claim 10, wherein the receptacle is a medical container configured to hold prescription drugs and the one or more first breath samples indicate a chemical breath profile of the user.

12. The method according to claim 10, wherein after the fastening unit securedly fixes the sealing apparatus to the receptacle, the user is permitted to input one or more second breath samples via the orifice.

13. The method according to claim 12, further comprising a comparison module for comparing the one or more second breath samples to the one or more first breath samples to obtain a result;
wherein the comparison module compares a plurality of select organic compounds detected in the breath samples and a concentration of each of the plurality of select organic compounds detected in the breath samples with predetermined organic compounds and predetermined ranges of concentration of select organic compounds.

14. The method according to claim 13, wherein the result either maintains the fastening unit in a locked state or actuates the fastening unit to an unlocked state.

15. The method according to claim 14, further comprising an indicator unit for indicating whether the fastening unit is in the locked state or in the unlocked state;
wherein the indicator unit is one or more of the following: a visual indicator, an audible indicator, or a combination of the visual and the audible indicators.

16. The method according to claim 10, wherein the analyzing module is a chemical breath component analyzer configured to:
(i) count each of a plurality of select organic compounds from the one or more first breath samples received from the user via the orifice; and
(ii) determine a concentration of each of the plurality of the select organic compounds.

17. The method according to claim 16, wherein each of the plurality of select organic compounds is selected based on one or more of the following: predetermined uncommon organic compounds, detected uncommon organic compounds, and uncommon concentrations of organic compounds.

18. The method according to claim 16, wherein the plurality of select organic compounds are extracted from one or more of the following: dead air space breath, alveolar breath, or a combination of dead air space breath and alveolar breath.

19. A method for manufacturing a sealing apparatus configured for use with a receptacle, the method comprising the steps of:
forming an orifice for receiving one or more first breath samples from a user;
constructing an analyzing module for analyzing the one or more first breath samples received from the user via the orifice;
constructing a storage unit for storing the one or more first breath samples; and
forming a fastening unit for securedly fixing the sealing apparatus to the receptacle once the one or more first breath samples have analyzed by the analyzing module and been stored in the storage unit.

20. A sealing apparatus, comprising:
an inlet for receiving a first breath sample from a user for fastening the sealing apparatus to an external device, the first breath sample including a plurality of organic compounds or molecules, a select combination of the plurality of organic compounds or molecules being used to create a unique breath profile; and
a module for analyzing and storing the first breath sample as the unique breath profile;
wherein the inlet further receives a second breath sample and compares the second breath sample with the first breath sample to determine whether access to the external device is permitted.
